(19) United States
(12) Patent Application Publication
(10) Pub. No.: US 2002/0052619 A1
(43) Pub. Date: May 2, 2002

(54) Dermal punches

(76) Inventor: James A. Transue, Bridgewater, NJ (US)

Correspondence Address: Steven B. Pokotilow, STROOCK & STROOCK & LAVAN LLP, 180 Maiden Lane, New York, NY, 10038, US

(21) Appl. No.: 09/880,252

(22) Filed: Jun. 13, 2001

Related U.S. Application Data
(60) Provisional application No. 60/211,292, filed on Jun. 13, 2000.

(51) Int. Cl. A61B 17/34

(52) U.S. Cl. 606/185

(57) ABSTRACT

A dermal punch blade adapted for use on a dermal punch having a handle, the blade having two opposed tapered concave arcuate sections and two opposed tapered convex arcuate sections, wherein four cutting edges disposed at the intersection of the two opposed concave sections and the two opposed convex sections.

### CROSS-REFERENCE TO RELATED APPLICATION

This application claims priority to Provisional Patent Application No. 60/211,292 filed on Jun. 13, 2000.

### FIELD OF THE INVENTION

The present invention is directed to an improved dermal punch device and methods of manufacturing and using the same.

### BACKGROUND OF THE INVENTION

In everyday medical practice, a commonly used instrument is the dermal punch. This is a relatively simple device used to extract a small area of skin in a manner not unlike a cookie cutter. They are generally circular or elliptical, come in a variety of sizes (diameters), and are generally available as either disposable or reusable.

The basic configuration of prior art disposable dermal punch devices are generally comprised of a handle, generally plastic and 3-4″ long, to which is rigidly mounted an approximately cylindrical blade coaxial with the handle. The blade, whether round or elliptical in sectional shape, is usually thin-wall metal tubing with a simple chamfered edge ground onto the outside of the business end to effect a cutting edge.

In use, such a blade is generally rotated by twisting the handle while exerting forward force in order to cut through the various layers of skin. It can be difficult to make a cut with such blades as the cutting edge is in a single plane and any forward force is diffused evenly along the entire edge. This is a common annoyance to clinicians and makes for a painful experience to the patient.

Many clinicians prefer elliptical punches because it is felt that the elliptical-shaped wound heals better cosmetically compared to a plain, circular incision. With an elliptical punch, a side-to-side rocking motion is generally imparted to the handle in order to assist with cutting. This is required for the same reason that the round punch must be rotated; the cutting edge lies in a single plane and any applied force is evenly diffused along the entire edge.

Examples of other prior art dermal punches are described in the following references. In U.S. Pat. No. 6,080,176 to Young (hereinafter “Young”), incorporated herein by reference, a medical punch 10 having several cutting edges is disclosed. As can be seen in FIGS. 8 and 9 of Young, the cutter 44b includes two concave arcuate cutting edges 57b and two flat projections 55b. Young does not disclose the method of forming a dermal punch by way of the specific grinding process, nor does Young disclose the specific turning and rocking motions of using the a punch in obtaining dermal samples.

U.S. Pat. No. 5,788,651 to Weilandt (hereinafter “Weilandt”), incorporated herein by reference, discloses an instrument for performing biopsies in which a cannula or 1 has teeth 12, 13, 14. As may be seen in FIG. 1, the teeth 12, 13, 14 of the cannula 1 form a serrated edge for cutting. In use, a finger tube 2, as shown in FIG. 2, with fingers 22 and 23 is passed over the canula with the fingers extending through windows 15 in order to sever the tissue plug as is shown in FIGS. 5D, E and F. The convex portions of Weilandt do not appear to be chamfered, and the concave portions appear to be chamfered on the inside, and not chamfered on the outside (see FIG. 3). Weilandt also does not disclose a method of forming a dermal punch by way of the specific grinding process, nor does Weilandt disclose the specific turning and rocking motions of using a dermal punch.

U.S. Pat. No. 6,027,458 to Janssens (hereinafter “Janssens”), incorporated herein by reference, discloses a device for taking a tissue sample having a knife 2 with cutting edges 7, as may be seen in FIG. 1. Janssens discloses the knife being used in conjunction with a receiving element 1 which extends coaxially and within knife 2 in order to obtain a tissue sample. Janssens discloses rotating the knife 2 so that cutting edges 7 may cut into and penetrate the tissue in order to obtain a sample. The cutting edges of Janssens, however, are only aligned to cut in one rotational direction, and Janssens does not disclose a blade with two opposed arcuate convex portions and two opposed arcuate concave portions, as the knife of Janssens has concave arcuate sections that meet at points, and not convex arcuate portions. Additionally, Janssens discloses a knife edge that is chamfered on the inside, and not the outside. Janssens also does not disclose the method of forming a dermal punch by way of the specific grinding process, nor the specific turning and rocking motions of using a dermal punch.

Accordingly, there is a need for an improved dermal punch that can enhance the cutting performance of the punch, as well as a method of manufacturing and using such a dermal punch.

### SUMMARY OF THE INVENTION

The present invention is directed to a dermal punch configuration that enhances the cutting performance of the punch while at the same time maintaining user familiarity and surgical technique. In addition, the present invention is directed to methods of manufacturing and using such an improved punch.

The dermal punch of the present invention comprises an elongated handle to which is rigidly mounted a thin-walled blade coaxially aligned with the handle. The blade of the dermal punch comprises two opposed tapered concave arcuate sections and two opposed tapered convex arcuate sections. Four cutting edges are disposed at the junctions of the two opposed concave edges and the two opposed convex edges. In a preferred embodiment, the blade has a generally circular or elliptical cutting edge configuration and is suited for cutting a sample of tissue with a rotary and/or rocking motion.

The present invention is also directed at methods of manufacturing such a dermal punch blade. In a preferred embodiment, a dermal punch is provided having an elongated handle to which is rigidly mounted a thin-walled, cylindrical blade, coaxially aligned with the handle. The blade has a circular or elliptical cutting edge configuration and is suited for cutting a sample of tissue with a rotary and/or rocking motion. The dermal punch is then fixed at an angle relative to horizontal that is slightly more than that of the preexisting chamfer of the blade. In a preferred embodiment, to form the blade of the present invention, a grinding wheel is used to grind a concave radius, somewhat larger than the radius of the punch blade, forming one portion of the cutting blade. Once the initial grind is completed, the punch is rotated 180° and a second grind is performed. The blade has a resultant shape having two opposed tapered concave arcuate sections and two opposed tapered convex arcuate sections.

Other objects and features of the present invention will become apparent from the following detailed description, considered in conjunction with the accompanying drawing figures. It is to be understood, however, that the drawings are designed solely for the purpose of illustration and not as a definition of the limits of the invention, for which reference should be made to the appended claims.

### BRIEF DESCRIPTION OF THE DRAWINGS

In the drawing figures, which are not to scale, and which are merely illustrative, and wherein like reference characters denote similar elements throughout the several views:

FIG. 1 is a perspective view of a dermal punch blade constructed in accordance with a preferred embodiment of the present invention;

FIG. 2 is a front elevational view of a dermal punch blade constructed in accordance with a preferred embodiment of the present invention;

FIG. 3 is a perspective view of the dermal punch blade of FIG. 2;

FIG. 4 is a side elevational view of the dermal punch blade of FIG. 2;

FIG. 5 is a bottom plan view of the dermal punch blade of FIG. 2;

FIG. 6 is a perspective view of a dermal punch blade constructed in accordance with an alterative preferred embodiment of the present invention;

FIG. 7 is a front elevational view of a dermal punch blade constructed in accordance with an alternative preferred embodiment of the present invention;

FIG. 8 is a perspective view of the dermal punch blade of FIG. 7;

FIG. 9 is a side elevational view of the dermal punch blade of FIG. 7;

FIG. 10 is a bottom plan view of the dermal punch blade of FIG. 7; and

FIG. 11 is a front elevational view depicting a method of manufacturing a dermal punch blade in accordance with a preferred embodiment of the present invention.

### DETAILED DESCRIPTION OF THE PRESENTLY PREFERRED EMBODIMENTS

The present invention is directed to a dermal punch configuration that greatly enhances the cutting performance of the punch while at the same time maintaining user familiarity and surgical technique. In addition, the present invention is directed to methods of manufacturing and using such an improved punch.

As seen in FIG. 2, the dermal punch, generally indicated as 8, of the present invention comprises an elongated handle 10 to which is mounted a thin-walled dermal punch blade 12 coaxially aligned with handle 10. In a preferred embodiment the blade 12 is rigidly mounted to handle 10. Blade 10 may be permanently mounted on handle 10, or may be removably mounted on handle 10, as a matter of application specific design choice. Thus, the dermal punch 8 may be disposable or, alternatively, handle 10 may be reusable with blade 12 being disposable.

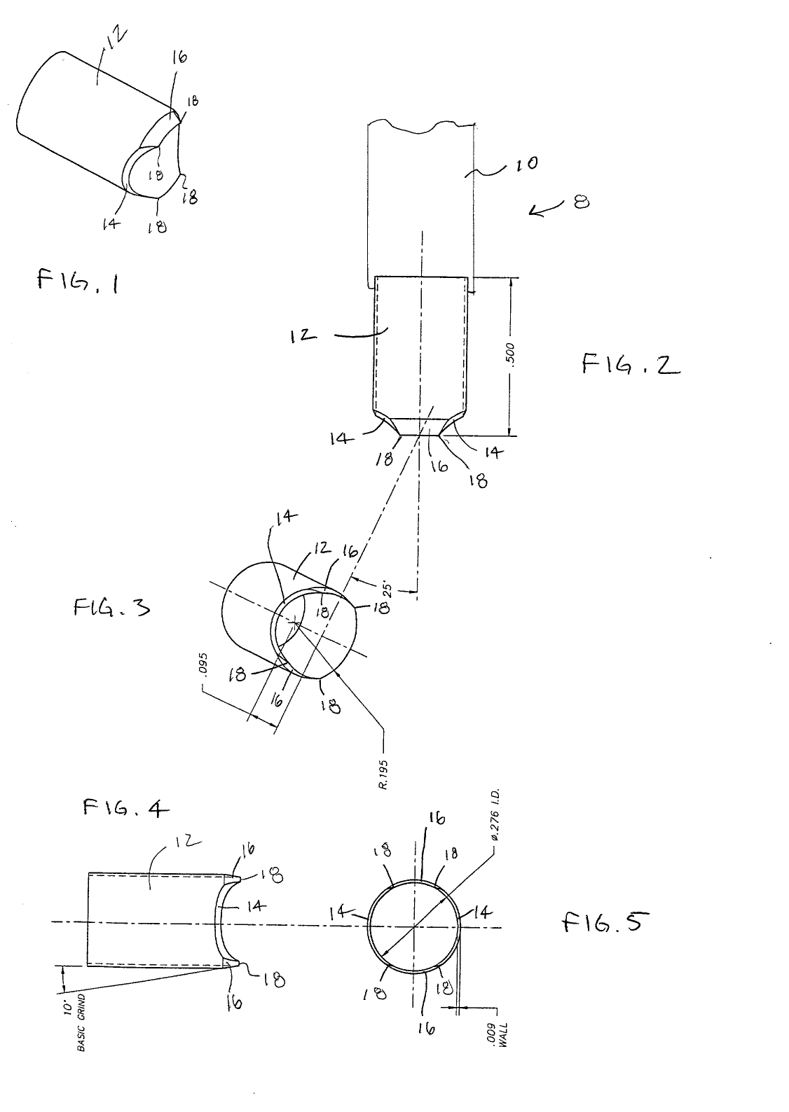

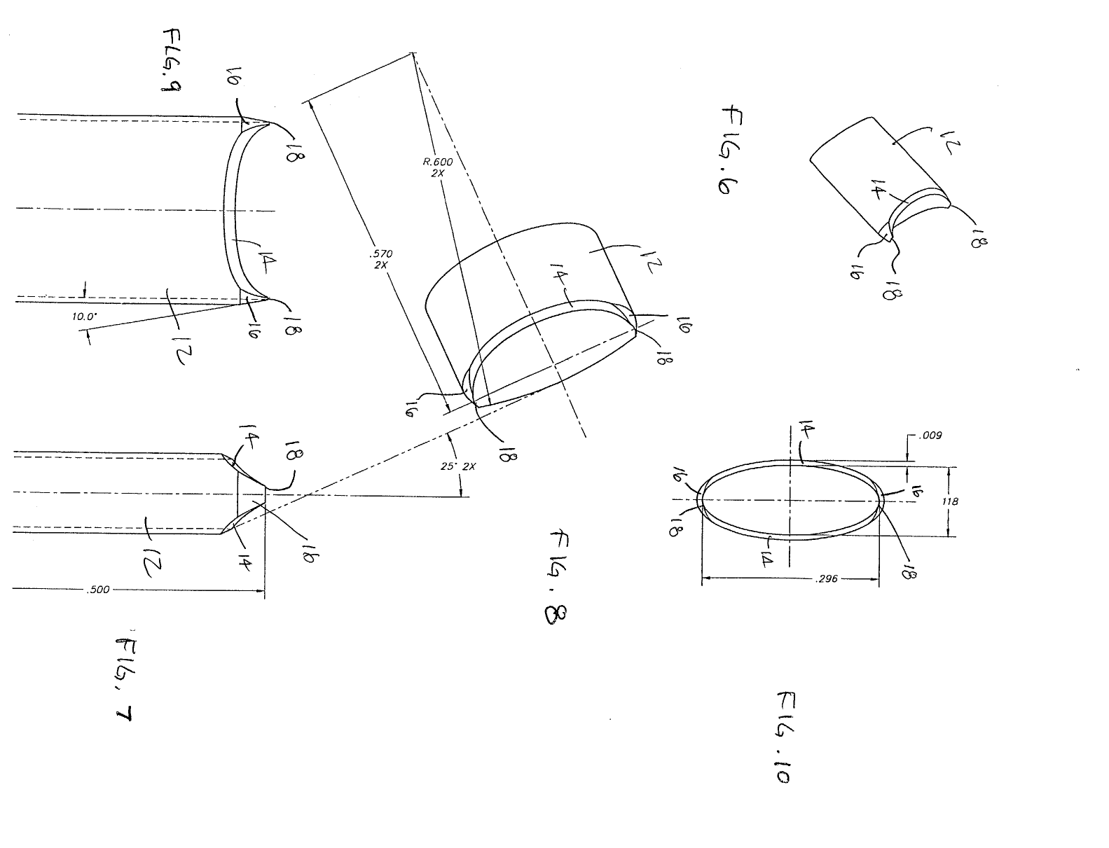

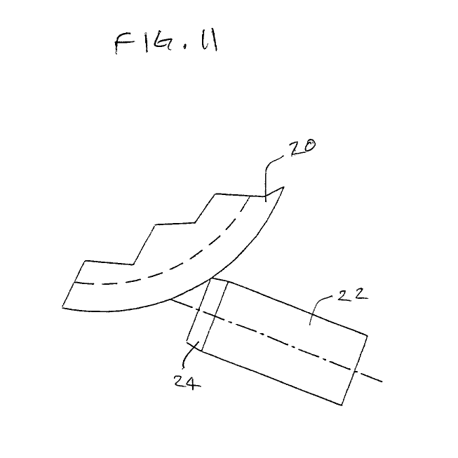

In a preferred embodiment, as seen for example in FIGS. 1-5, blade 12 of the dermal punch comprises two opposed tapered (or chamfered) concave arcuate sections 14 and two opposed tapered convex arcuate sections 16. Four cutting edges 18 are disposed at the junctions of the two opposed concave sections 14 and the two opposed convex sections 16. In a preferred embodiment, as seen in FIGS. 1-5, blade 12 has a generally circular cross section. In an alternative preferred embodiment, as seen in FIGS. 6-10, blade 12 has a generally elliptical cross section. In each configuration, the blade 12 is suited for cutting a sample of tissue with a rotating and/or rocking motion.

As seen, for example in FIGS. 1 and 6, in a preferred embodiment of the present invention concave sections 14 and the convex sections 16 of blade 12 are each tapered or chamfered on the outside edges, while the inside edges are not chamfered. In the preferred embodiment depicted in FIGS. 1-5, the longitudinal length of the blade 12 is approximately 0.5 inches, the blade is generally circular in cross section, the inner diameter of the blade is approximately 0.276 inches, and the wall of blade 12 is approximately 0.009 inches thick. Also, as seen in FIG. 5, the length of the concave sections 14 is longer than that of the convex sections 16. In the preferred embodiment depicted in FIGS. 6-10, the longitudinal length of the blade 12 is approximately 0.5 inches, the blade is generally elliptical in cross section, the inside dimension of the blade along the short axis is approximately 0.118 inches and 0.296 in along the long axis, and the wall of blade 12 is approximately 0.009 inches thick.

The present invention is also directed at methods of manufacturing and using such a dermal punch blade. The dermal punch of the present invention greatly enhances cutting performance while maintaining user familiarity and surgical technique. In addition, the blade of the present invention is easily manufactured by requiring the additional of only two relatively simple operations beyond existing configurations. That is, as described below, by beginning with a conventional dermal punch blade, two additional side grinds of a very specific geometry yield a totally different cutting mechanism.

In order to manufacture the blade of the present invention, in a preferred embodiment, a conventional round punch blade 22 is fixed about its axis at an angle relative to horizontal that is slightly more than that of the pre-existing chamfer 24 of the blade 22 (see FIG. 11). For example, if the basic chamfer is 10° then the blade 22 will be positioned at 20° above horizontal. In this position, a grinding wheel is then traversed inline with the punch axis. This grinding wheel has a profile dressed onto its periphery. The profile is a concave radius somewhat larger than the radius of the punch. As an example, the radius of the profile can be equal to the diameter of the punch. Approximations can be attempted with a straight, rather than radiused, grinding wheel but the four cutting edges are not as optimal. The same manufacturing method can apply to both round and elliptical punches. Once the above grind is completed, the blade 22 is rotated 180° about its axis and the grind is duplicated. In a preferred embodiment, post-grind electropolishing is applied to remove any grinding burrs, enhance sharpness, and provide a smoother surface and a passivation oxide layer for corrosion resistance.

The blade 12 has a resultant shape having two opposed tapered concave arcuate sections 14 and two opposed tapered convex arcuate sections 16. Such a blade produces four cutting edges 18: two which cut when said blade is rotated in a clockwise rotation and two of which cut only when the blade is rotated in a counterclockwise direction. The result is a blade capable of concentrating forces at cutting edges that are very nearly perpendicular to the direction of rotation with an extremely sharp, shallow-angled junction between the new grind and the pre-existing chamfer. The remnants of the original chamfer serve to sever the tissue plug subcutaneously by rocking the device back and forth.

The creation of physical features on a cutting edge, to provide a “steak-knife” effect, are well known and can be accomplished by a variety of relatively complex methods. The present invention utilizes a relative simple manufacturing method to yield a unique cutting edge that is better suited for cutting a plug of tissue with a rotary motion.

Thus, while there have been shown and described and pointed out novel features of the present invention as applied to preferred embodiments thereof, it will be understood that various omissions and substitutions and changes in the form and details of the disclosed invention may be made by those skilled in the art without departing from the spirit of the invention. It is the intention, therefore, to be limited only as indicated by the scope of the claims appended hereto.

It is also to be understood that the following claims are intended to cover all of the generic and specific features of the invention herein described and all statements of the scope of the invention which, as a matter of language, might be said to fall there between. In particular, this invention should not be construed as being limited to the dimensions, proportions or arrangements disclosed herein.

What is claimed is:

1. A dermal punch blade adapted for use on a dermal punch having a handle, said blade comprising two opposed tapered concave arcuate sections and two opposed tapered convex arcuate sections, and having four cutting edges disposed at the intersection of the two opposed concave sections and the two opposed convex sections.

2. The dermal punch blade according to claim 1, wherein two of said four cutting edges cut tissue when said blade is rotated in a clockwise direction, and the other two of said four cutting edges cut tissue when said blade is rotated in a counterclockwise direction.

3. The dermal punch blade according to claim 1, wherein said blade has a circular or elliptical cross section.

4. The dermal punch blade according to claim 1, wherein said concave and convex sections are chamfered on an inside edge thereof.

5. The dermal punch blade according to claim 1, wherein said concave and convex sections are chamfered only on an inside edge thereof.

6. The dermal punch blade according to claim 1, wherein said blade is circular in cross section and has an inner diameter of approximately 0.27 inches, a wall thickness of approximately 0.09 inches, and a longitudinal length of approximately 0.5 inches.

7. The dermal punch blade according to claim 1, wherein said blade is elliptical in cross section and has an inside dimension of approximately 0.12 inches along the short axis, an inside dimension of approximately 0.29 inches along the short axis, a wall thickness of approximately 0.09 inches, and a longitudinal length of approximately 0.5 inches.

8. The dermal punch blade according to claim 1, wherein the length of said concave sections exceeds that of said convex sections.

9. The dermal punch blade according to claim 1, wherein said blade is adapted to be coaxially aligned with said handle.

10. A method of manufacturing a dermal punch blade comprising two opposed tapered concave arcuate sections and two opposed tapered convex arcuate sections, comprising the steps of:
(a) positioning a punch blade about its axis at an angle relative to horizontal that is slightly more than that of a pre-existing chamfer of said blade;
(b) transversing a grinding wheel having a profile on its periphery inline with the punch blade axis to grind at least a portion of said punch blade;
(c) rotating said punch blade about its axis approximately 180°; and
(d) repeating step (b).

11. The method according to claim 10, wherein said blade has a chamfer of approximately 10° and said blade is positioned at approximately 20° above horizontal.

12. The method according to claim 10, wherein said profile is a concave radius larger than that of said punch blade.

13. The method according to claim 10, wherein said profile has a radius approximately equal to the diameter of said punch blade.

14. The method according to claim 10, wherein said punch blade is a conventional round or elliptical punch blade.

15. The method according to claim 10, further comprising the step of electropolishing said punch blade.

16. A method of using a dermal punch blade mounted on a handle and comprising two opposed tapered concave arcuate sections and two opposed tapered convex arcuate sections to cut a sample of tissue, comprising the steps of:
(a) positioning said dermal punch blade against said tissue;
(b) rotating said handle clockwise to cause two sections of said blade to cut said tissue; and
(c) rotating said handle counterclockwise to cause the other two sections of said blade to cut said tissue.

17. The method according to claim 16, wherein said concave and convex sections of said blade each have a chamfer on an outside edge thereof, and further comprising the step of using said chamfer to sever said tissue subcutaneously.
